(19) United States
(12) Patent Application Publication
(10) Pub. No.: US 2014/0330155 A1
(43) Pub. Date: Nov. 6, 2014

(54) METHOD AND APPARATUS FOR MONITORING AND CONTROLLING A PRESSURE SUPPORT DEVICE

(71) Applicant: KONINKLIJKE PHILIPS N.V., EINDHOVEN (NL)

(72) Inventors: Lara Marie Brewer, Bountiful, UT (US); Joseph Allen Orr, Park City, UT (US)

(21) Appl. No.: 14/366,014

(22) PCT Filed: Dec. 21, 2012
(86) PCT No.: PCT/IB2012/057601

§ 371 Date: Jun. 17, 2014

Related U.S. Application Data
(60) Provisional application No. 61/579,722, filed on Dec. 23, 2011.

(51) Int. Cl. A61B 5/08; A61B 5/087

(52) U.S. Cl. CPC A61B 5/0826; A61B 5/082; A61B 5/087
USPC 600/532

(57) ABSTRACT

A system and method are configured to monitor respiratory stability and/or effectiveness. A ventilation index is determined that provides an indication of respiratory stability and/or effectiveness. The ventilation index is determined from one or more metrics that indicate deviation of a breathing parameter of a subject from levels that have been observed to be normal or typical for the subject.

### BACKGROUND

1. Field

The present disclosure pertains to a method and apparatus for monitoring respiration of a subject by determining a ventilation index that indicates respiratory stability and/or effectiveness.

2. Description of the Related Art

It is well known to indices that provide an indication of respiratory stability and/or effectiveness. However, conventional indices typically fail to provide one or more of (i) proactive alarms that indicate respiratory issues before full onset, (ii) meaningful indication of respiratory stability and/or effectiveness relying solely on gas parameter detection (e.g., capnography), (iii) an indication of respiratory stability and/or effectiveness that is based on previous respiration of a subject being monitored, and/or have other shortcomings

### SUMMARY

Accordingly, one or more aspects of the present disclosure relate to a system configured to monitor respiration of a subject. In some embodiments, the system comprises one or more gas parameter sensors and a processor. The gas parameter sensors are configured to generate output signals conveying information related to one or more gas parameters in a respiratory circuit. The respiratory circuit comprises a subject interface appliance configured to communicate with the airway of a subject. The processor is configured to execute computer program modules, the computer program modules comprising a breathing parameter module, a respiratory rate monitor module, an apnea monitor module, an end tidal carbon dioxide monitor module, and a ventilation index monitor module. The breathing parameter module is configured to determine breathing parameters of the respiration of the subject based on the output signals, the breathing parameters comprising (i) a first parameter related to breath length, and (ii) a second parameter related to end tidal carbon dioxide. The respiratory rate monitor module is configured to determine, in an ongoing manner, a rate metric based on a comparison of the first parameter for a first set of breaths by the subject with the first parameter for a first subset of one or more breaths, wherein the one or more breaths in the first subset of one or more breaths are also in the first set of breaths by the subject. The apnea monitor module is configured to determine, in an ongoing manner based on the output signals, an apnea metric that represents whether the subject is currently experiencing an apnea, and, responsive to the subject currently experiencing an apnea, a severity and/or duration of the apnea. The end tidal carbon dioxide monitor module is configured to determine, in an ongoing manner, an end tidal carbon dioxide metric based on a comparison of the second parameter for a second set of breaths by the subject with the second parameter for a second subset of one or more breaths, wherein the one or more breaths in the second subset of one or more breaths are also in the second set of breaths by the subject. The ventilation index module is configured to determine, in an ongoing manner, a ventilation index for the subject based on the rate metric, the apnea metric, and the end tidal carbon dioxide metric, such that the ventilation index at a given time represents respiratory stability and/or effectiveness for the subject at the given time.

Yet another aspect of the present disclosure relates to a method of monitoring respiration of a subject. In some embodiments, the method comprises receiving output signals conveying information related to one or more gas parameters in a respiratory circuit, wherein the respiratory circuit comprises a non-invasive subject interface appliance configured to non-invasively communicate with the airway of a subject; determining breathing parameters of the respiration of the subject based on the output signals, the breathing parameters comprising (i) a first parameter related to breath length, and (ii) a second parameter related to end tidal carbon dioxide; determining, in an ongoing manner, a rate metric based on a comparison of the first parameter for a first set of breaths by the subject with the first parameter for a first subset of one or more breaths, wherein the one or more breaths in the first subset of one or more breaths are also in the first set of breaths by the subject; determining, in an ongoing manner based on the output signals, an apnea metric that represents whether the subject is currently experiencing an apnea, and, responsive to the subject currently experiencing an apnea, a severity and/or duration of the apnea; determining, in an ongoing manner, an end tidal carbon dioxide metric based on a comparison of the second parameter for a second set of breaths by the subject with the second parameter for a second subset of one or more breaths, wherein the one or more breaths in the second subset of one or more breaths are also in the second set of breaths by the subject; and determining, in an ongoing manner, a ventilation index for the subject based on the rate metric, the apnea metric, and the end tidal carbon dioxide metric, such that the ventilation index at a given time represents respiratory stability and/or effectiveness for the subject at the given time.

Still another aspect of present disclosure relates to a system configured to monitor respiration of a subject. In some embodiments, the method comprises means for receiving output signals conveying information related to one or more gas parameters in a respiratory circuit, wherein the respiratory circuit comprises a subject interface appliance configured to communicate with the airway of a subject; means for determining breathing parameters of the respiration of the subject based on the output signals, the breathing parameters comprising (i) a first parameter related to breath length, and (ii) a second parameter related to end tidal carbon dioxide; means for determining, in an ongoing manner, a rate metric based on a comparison of the first parameter for a first set of breaths by the subject with the first parameter for a first subset of one or more breaths, wherein the one or more breaths in the first subset of one or more breaths are also in the first set of breaths by the subject; means for determining, in an ongoing manner based on the output signals, an apnea metric that represents whether the subject is currently experiencing an apnea, and, responsive to the subject currently experiencing an apnea, a severity and/or duration of the apnea; means for determining, in an ongoing manner, an end tidal carbon dioxide metric based on a comparison of the second parameter for a second set of breaths by the subject with the second parameter for a second subset of one or more breaths, wherein the one or more breaths in the second subset of one or more breaths are also in the second set of breaths by the subject; and means for determining, in an ongoing manner, a ventilation index for the subject based on the rate metric, the apnea metric, and the end tidal carbon dioxide metric, such that the ventilation index at a given time represents respiratory stability and/or effectiveness for the subject at the given time.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a system configured to monitor respiration of a subject; and

FIG. 2 is a method of monitoring respiration of a subject.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

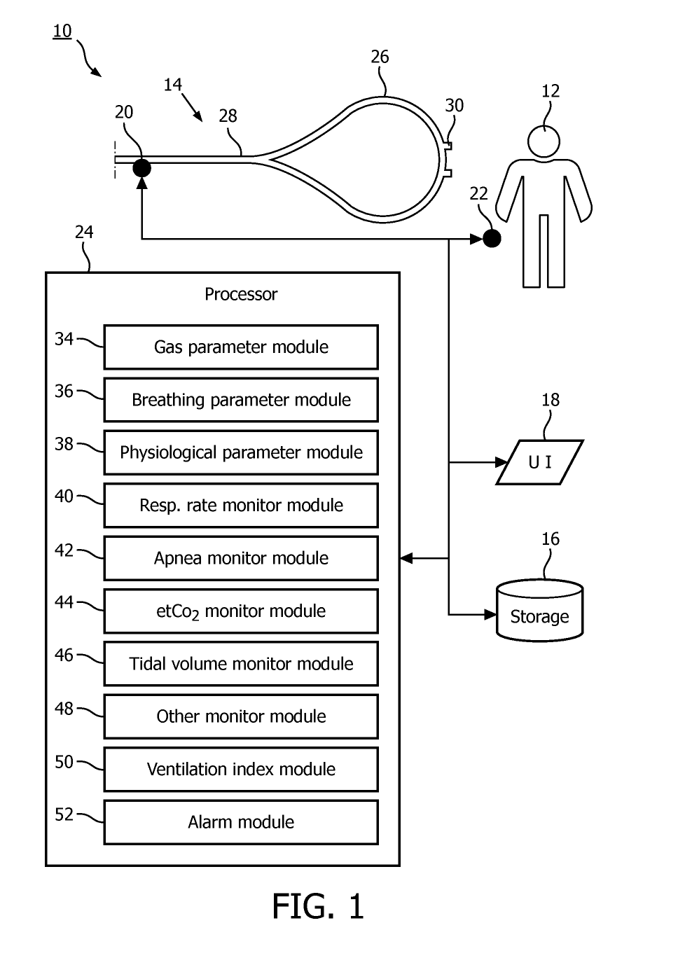

FIG. 1 illustrates a system 10 configured to monitor respiration of a subject 12. Such monitoring may include one or more of scoring respiratory stability and/or effectiveness, recommending suggestions of adjustments to a respiratory therapy being provided to subject 12, identifying specific issues with the respiration of subject 12, and/or other monitoring. In one embodiment, system 10 includes a respiratory circuit 14, electronic storage 16, a user interface 18, one or more gas parameter sensors 20, one or more physiological sensors 22, a processor 24, and/or other components.

Respiratory circuit 14 is configured to interface with the airway of subject 12. The pressurized flow of breathable gas is delivered to the airway of subject 12 via a subject interface 26. Subject interface 26 is configured to provide fluid communication with the airway of subject 12. As such, subject interface 26 includes a conduit 28 and an interface appliance 30. Conduit 28 conveys the gas to and/or from interface appliance 30, and interface appliance 30 places conduit 28 in communication with the airway of subject 12. In some embodiments, the subject interface 26 is non-invasive. As such, interface appliance 30 non-invasively engages subject 12. Non-invasive engagement includes removably engaging an area (or areas) surrounding one or more external orifices of the airway of subject 12 (e.g., nostrils and/or mouth) to communicate gas between the airway of subject 12 and subject interface 26. Some examples of non-invasive interface appliance 30 may include, for example, a nasal cannula, a nasal mask, a nasal/oral mask, a full face mask, a total face mask, or other interface appliances that communicate a flow of gas with an airway of a subject. In some embodiments, interface appliance 30 is invasive. Examples of an invasive interface appliance include an endotracheal tube, laryngeal mask airway, and/or other invasive interface appliances.

In some embodiments, electronic storage 16 comprises electronic storage media that electronically stores information. The electronic storage media of electronic storage 16 may include one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 16 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 16 may store software algorithms, information determined by processor 24, information received via user interface 18, and/or other information that enables system 10 to function properly. Electronic storage 16 may be (in whole or in part) a separate component within system 10, or electronic storage 16 may be provided (in whole or in part) integrally with one or more other components of system 10 (e.g., user interface 18, processor 24, etc.).

User interface 18 is configured to provide an interface between system 10 and one or more users (e.g., subject 12, a caregiver, a researcher, a therapy decision-maker, etc.) through which the users may provide information to and receive information from system 10. This enables data, cues, results, and/or instructions and any other communicable items, collectively referred to as “information,” to be communicated between the users and one or more of the pressure generator, electronic storage 16, and/or processor 24. Examples of interface devices suitable for inclusion in user interface 18 include a keypad, buttons, switches, a keyboard, knobs, levers, a display screen, a touch screen, speakers, a microphone, an indicator light, an audible alarm, a printer, a tactile feedback device, and/or other interface devices. In one embodiment, user interface 18 includes a plurality of separate interfaces.

It is to be understood that other communication techniques, either hard-wired or wireless, are also contemplated by the present invention as user interface 18. For example, the present invention contemplates that user interface 18 may be integrated with a removable storage interface provided by electronic storage 16. In this example, information may be loaded into system 10 from removable storage (e.g., a smart card, a flash drive, a removable disk, etc.) that enables the user(s) to customize the implementation of system 10. Other exemplary input devices and techniques adapted for use with system 10 as user interface 18 include, but are not limited to, an RS-232 port, RF link, an IR link, modem (telephone, cable or other). In short, any technique for communicating information with system 10 is contemplated by the present invention as user interface 18.

Gas parameter sensors 20 are configured to generate output signals conveying information related to one or more gas parameters of the gas within subject interface 26. The one or more gas parameters may include, for example, flow, volume, pressure, composition or concentration (e.g., level(s) of one or more molecular species, such as carbon dioxide, oxygen, medicaments, and/or other molecular species), and/or other gas parameters. Gas parameter sensors 20 may include one or more sensors that measure such parameters directly (e.g., through fluid communication with the pressurized flow of breathable gas at the pressure generator or in subject interface 26). Gas parameter sensors 20 may include one or more sensors that generate output signals related to one or more parameters of the pressurized flow of breathable gas indirectly. For example, one or more of sensors 20 may generate an output based on an operating parameter of the pressure generator (e.g., a valve driver or motor current, voltage, rotational velocity, and/or other operating parameters), and/or other sensors. Although gas parameter sensors 20 are illustrated at a single location at or adjacent to an interface between interface appliance 30 and conduit 28, this is not intended to be limiting. Gas parameter sensors 20 may include sensors disposed in a plurality of locations, such as for example, within the pressure generator, within (or in communication with) conduit 28, within (or in communication with) interface appliance 30, within an exhaust conduit (not shown), in a sidestream configuration (e.g., receiving a flow of breathable gas for measurement from conduit 28), and/or other locations.

Physiological sensors 22 include one or more of sensors configured to generate output signals conveying information related to one or more physiological parameters of subject 12, other than gas parameters detected by gas parameter sensors 20. Such parameters may include, for example, one or more of oxygen saturation, other blood gas levels, pulse rate, pulse shape, pulse transit time, pulse pressure variation, delta pulse pressure, delta down, respiratory effort, and/or other physiological parameters. In some embodiments, one or more of physiological sensors 22 are configured to provide the output signals to processor 24. In some embodiments, one or more of physiological sensors 22 are configured such that a user reads a measurement made by the sensor, and inputs the measurement to system 10 manually (e.g., through user interface 18).

Processor 24 is configured to provide information processing capabilities in system 10. As such, processor 24 may include one or more of a digital processor, an analog processor, a digital circuit designed to process information, an analog circuit designed to process information, a state machine, and/or other mechanisms for electronically processing information. Although processor 24 is shown in FIG. 1 as a single entity, this is for illustrative purposes only. In some implementations, processor 24 may include a plurality of processing units. These processing units may be physically located within the same device (e.g., pressure generator 14), or processor 24 may represent processing functionality of a plurality of devices operating in coordination. For example, processor 24 may represent a first processor (or processors) within a ventilator including pressure generator 14 and a second processor within a gas analysis device or system (e.g., a patient monitor) that is separate from the ventilator.

As is shown in FIG. 1, processor 24 may be configured to execute one or more computer program modules. The one or more computer program modules may include one or more of a gas parameter module 34, a breathing parameter module 36, a physiological parameter module 38, a respiratory rate monitor module 40, an apnea monitor module 42, an end tidal carbon dioxide monitor module 44, a tidal volume monitor module 46, one or more other monitor modules 48, ventilation index module 50, alarm module 52, and/or other modules. Processor 24 may be configured to execute modules 34, 36, 38, 40, 42, 44, 46, 48, 50, and/or 52 by software; hardware; firmware; some combination of software, hardware, and/or firmware; and/or other mechanisms for configuring processing capabilities on processor 24.

It should be appreciated that although modules 34, 36, 38, 40, 42, 44, 46, 48, 50, and 52 are illustrated in FIG. 1 as being co-located within a single processing unit, in implementations in which processor 24 includes multiple processing units, one or more of modules 34, 36, 38, 40, 42, 44, 46, 48, 50, and/or 52 may be located remotely from the other modules. The description of the functionality provided by the different modules 34, 36, 38, 40, 42, 44, 46, 48, 50, and/or 52 described below is for illustrative purposes, and is not intended to be limiting, as any of modules 34, 36, 38, 40, 42, 44, 46, 48, 50, and/or 52 may provide more or less functionality than is described. For example, one or more of modules 34, 36, 38, 40, 42, 44, 46, 48, 50, and/or 52 may be eliminated, and some or all of its functionality may be provided by other ones of modules 34, 36, 38, 40, 42, 44, 46, 48, 50, and/or 52. As another example, processor 24 may be configured to execute one or more additional modules that may perform some or all of the functionality attributed below to one of modules 34, 36, 38, 40, 42, 44, 46, 48, 50, and/or 52.

Gas parameter module 34 is configured to determine one or more gas parameters of the flow of breathable gas within subject interface 26 based on the output signals generated by gas parameter sensors 20. The one or more gas parameters may include, for example, one or more of a pressure, a flow rate, a volume, a composition or concentration (e.g., a partial pressure of a molecular species, an amount of a molecular species, relative level of a molecular species, and/or other information related to composition), a temperature, a humidity, and/or other gas parameters. The determination of the one or more gas parameters by gas parameter module 34 is ongoing over time. The “ongoing” determination of a gas parameter (and/or other parameters, indices, metrics, scores, etc.) may refer to individual determinations of the gas parameter at different times as time goes on. The different times may be determined, for example, by a sampling rate.

Breathing parameter module 36 is configured to determine breathing parameters of the respiration of subject 12 based on the output signals generated by gas parameter sensors 20. For example, breathing parameter module 36 may be configured to determine the breathing parameters based on, for example, the gas parameters determined by gas parameter module 34, and/or directly from the output signals generated by gas parameter sensors 20. The breathing parameters determined by breathing parameter module 36 may include one or more of a respiratory rate, a breath time, an inhalation time, an exhalation time, end-exhalation pause time, end tidal carbon dioxide (e.g., amount, partial pressure, etc.), a inspiratory tidal volume, a expiratory tidal volume, carbon dioxide volume excretion, and/or other breathing parameters. Breathing parameter module 36 is configured to determine the breathing parameters in an ongoing manner (e.g., at a sampling rate, on a per-breath basis, and/or at other intervals).

Physiological parameter module 38 is configured to determine one or more physiological parameters based on the output signals generated by physiological sensors 22. The physiological parameters may be parameters different from the breathing parameters determined by breathing parameter module 36. Without limitation, the one or more physiological parameters may include one or more of oxygen saturation, other blood gas levels, pulse rate, pulse shape, pulse transit time, pulse pressure variation, delta pulse pressure, delta down, respiratory effort, and/or other physiological parameters.

Respiratory rate monitor module 40 is configured to determine a rate metric that indicates deviation of breath time and/or respiratory rate from a typical breath time or respiratory rate of subject 12 (e.g., as determined by gas parameter module 34). In other words, the rate metric provides an indication of whether subject 12 respiratory rate has deviated from the normal rate/breath time of subject 12. Using breath time to determine the rate metric, the inverse of respiratory rate (1/RR), may provide a more accurate indication of deviations because there is a bigger difference between a respiratory rate of 6 breaths a minute and 4 breaths a minute (10 seconds per breath vs. 15 seconds per breath) than there is between a respiratory rate of 14 breaths per minute and 12 breaths per minute (4.3 seconds per breath vs. 5 seconds per breath). The rate metric may be a score, an amount of time, a condition rating (e.g., red-yellow-green, good-medium-bad, and/or other rating systems), and/or other metrics.

In some embodiments, respiratory rate monitor module 40 is configured such that the rate metric is determined in an ongoing manner (e.g., on a per-breath basis) based on a moving window within which the average and standard deviation of breath time are calculated for a set of breaths of subject 12 taken within the moving window of time. For example, a z-score for a subset of one or more breaths is calculated as a comparison of the breath time for the subset of one or more breaths with the values of standard deviation and average of breath time for the set of breaths that transpired within the moving window. The length of time that corresponds to the moving window may be a configurable setting (e.g., by a caregiver), or may be pre-defined without the possibility of customization. The z-score may be implemented as the rate metric, and/or may be implemented in the determination of the rate metric. In some embodiments, the z-score is compared with a threshold, and the rate metric indicates a relationship of the z-score to the threshold. For example, the rate metric may indicate an amount of time (or number of breaths) the z-score has remained above or below the threshold, how many times the z-score has crossed the threshold over a period of time (or number of breaths), and/or may indicate other information about the rate metric with respect to the threshold. The threshold may be determined based on historical information associated with subject 12 (e.g., previous data collected by system 10), one or more user-configurable settings, pre-defined, and/or determined based on other information. As such, the rate metric may indicate the presence of or high likelihood of upcoming respiratory depression. By way of non-limiting example, in some embodiments, an event of onset of respiratory depression is defined as five or more subsequent breath times which are longer than 1.8×the standard deviation for subject 12 (z-score>1.8).

Apnea monitor module 42 is configured to determine an apnea metric that indicates whether subject 12 is currently experiencing an apnea. Responsive to subject 12 currently experiencing an apnea, the apnea metric may indicate a severity and/or duration of the current apnea. Apnea monitor module 42 is configured to determine the apnea metric based on the output signals generated by gas parameter sensors 20. This may include determining the apnea metric based on one or more of the gas parameters determined by gas parameter module 34 (e.g., pressure and/or flow of the flow of breathable gas). Apnea monitor module 42 is configured to determine the apnea metric in an ongoing manner. The apnea metric may be a score, an amount of time, a condition rating (e.g., red-yellow-green, good-medium-bad, and/or other rating systems), and/or other metrics.

End tidal carbon dioxide monitor module 44 is configured to determine a end tidal carbon dioxide metric that indicates deviation of end tidal carbon dioxide (e.g., partial pressure and/or amount) from a typical end tidal carbon dioxide or carbon dioxide excretion volume of subject 12 (e.g., as determined by gas parameter module 34). In other words, the end tidal carbon dioxide or carbon dioxide excretion volume metric provides an indication of whether end tidal carbon dioxide for subject 12 has deviated from the normal end tidal carbon dioxide or carbon dioxide excretion volume of subject 12. The end tidal carbon dioxide metric may be a score, an amount of time, a condition rating (e.g., red-yellow-green, good-medium-bad, and/or other rating systems), and/or other metrics.

In some embodiments, end tidal carbon dioxide monitor module 44 is configured such that the end tidal carbon dioxide metric is determined in an ongoing manner (e.g., on a per-breath basis) based on a moving window within which the average and standard deviation of end tidal carbon dioxide (and/or some other measurement related to end tidal carbon dioxide, such as volumetric carbon dioxide, and/or other measurements) are calculated for a set of breaths of subject 12 taken within the moving window of time. For example, a z-score for a subset of one or more breaths is calculated as a comparison of the end tidal carbon dioxide for the subset of one or more breaths with the values of standard deviation and average of end tidal carbon dioxide for the set of breaths that transpired within the moving window. The length of time that corresponds to the moving window may be a configurable setting (e.g., by a caregiver), or may be pre-defined without the possibility of customization. The length of time may be the same (or different) as corresponds to the length of time for the moving window implemented by respiratory rate monitor module 40. The z-score may be implemented as the end tidal carbon dioxide metric, and/or may be implemented in the determination of the end tidal carbon dioxide metric. In some embodiments, the z-score is compared with a threshold, and the end tidal carbon dioxide metric indicates a relationship of the z-score to the threshold. For example, the end tidal carbon dioxide metric may indicate an amount of time (or number of breaths) the z-score has remained above or below the threshold, how many times the z-score has crossed the threshold over a period of time (or number of breaths), and/or may indicate other information about the end tidal carbon dioxide metric with respect to the threshold. The threshold may be determined based on historical information associated with subject 12 (e.g., previous data collected by system 10), one or more user-configurable settings, pre-defined, and/or determined based on other information.

Tidal volume monitor module 46 is configured to determine a tidal volume metric that indicates deviation of tidal volume from a typical tidal volume of subject 12 (e.g., as determined by gas parameter module 34). In other words, the tidal volume metric provides an indication of whether tidal volume for subject 12 has deviated from the normal tidal volume of subject 12. The tidal volume metric may be a score, an amount of time, a condition rating (e.g., red-yellow-green, good-medium-bad, and/or other rating systems), and/or other metrics.

In some embodiments, tidal volume monitor module 46 is configured such that the tidal volume metric is determined in an ongoing manner (e.g., on a per-breath basis) based on a moving window within which the average and standard deviation of tidal volume are calculated for a set of breaths of subject 12 taken within the moving window of time. For example, a z-score for a subset of one or more breaths is calculated as a comparison of the tidal volume for the subset of one or more breaths with the values of standard deviation and average of tidal volume for the set of breaths that transpired within the moving window. The length of time that corresponds to the moving window may be a configurable setting (e.g., by a caregiver), or may be pre-defined without the possibility of customization. The length of time may be the same (or different) as corresponds to the length of time for the moving window implemented by respiratory rate monitor module 40 and/or end tidal carbon dioxide monitor module 44. The z-score may be implemented as the tidal volume metric, and/or may be implemented in the determination of the tidal volume metric. In some embodiments, the z-score is compared with a threshold, and the tidal volume metric indicates a relationship of the z-score to the threshold. For example, the tidal volume metric may indicate an amount of time (or number of breaths) the z-score has remained above or below the threshold, how many times the z-score has crossed the threshold over a period of time (or number of breaths), and/or may indicate other information about the tidal volume metric with respect to the threshold. The threshold may be determined based on historical information associated with subject 12 (e.g., previous data collected by system 10), one or more user-configurable settings, pre-defined, and/or determined based on other information.

Other monitor module 48 is configured to determine one or more other metrics. One or more of the other metrics may be determined based on output signals generated by gas parameter sensors 20 and/or physiological sensors 22. The metrics may convey information related to respiratory and/or physiological parameters other than respiratory rate, apneas, end tidal carbon dioxide, volumetric capnography, and/or tidal volume. Such parameters may include, for example, ECG, heart rate, arterial pressure waveform, oxygen saturation, and/or other parameters.

Ventilation index module 50 is configured to determine a ventilation index for the subject in an ongoing manner. The ventilation index represents the respiratory stability and/or effectiveness of subject 12. Ventilation index module 50 is configured to determine the ventilation index based on one or more of the rate metric, the apnea metric, the end tidal carbon dioxide metric, the tidal volume metric, and/or one or more of the other metrics. The ventilation index may be determined according to one or more mathematical algorithms using numerical metrics as inputs. The ventilation index may be determined from a look-up table that uses the appropriate metrics as inputs. In such embodiments, ventilation index module 50 may be configured to perform a mapping of the metrics used as inputs to an appropriate index output. The ventilation index may be a score, an amount of time, a condition rating (e.g., red-yellow-green, good-medium-bad, and/or other rating systems), and/or other indices.

Alarm module 52 is configured generate one or more alarms based on the ventilation index. The generation of an alarm may be based, for example, on a comparison of the ventilation index with a threshold, an observation of the frequency of the index value within a defined range, mapping of the ventilation index to an alarm, and/or other techniques. Comparing the ventilation index with a threshold may include determining an amount of time (or number of breaths) for which the ventilation has crossed the threshold. Responsive to an amount of time (or number of breaths) reaching some pre-determined amount, an alarm may be generated. The threshold, and/or the pre-determined amount of time may be user configurable (e.g., based on user settings), pre-defined without the opportunity for customization, determined automatically (e.g., based on data gathered by system 10 during usage by subject 12), and/or determined in other ways. The alarms may provide insight with respect to a probable problem with subject 12 and/or system 10, a suggested action to be performed to address the alarm (e.g., to be taken by subject 12 and/or a caregiver), and/or other insights. Alarms generated by alarm module 52 may be presented via user interface 18.

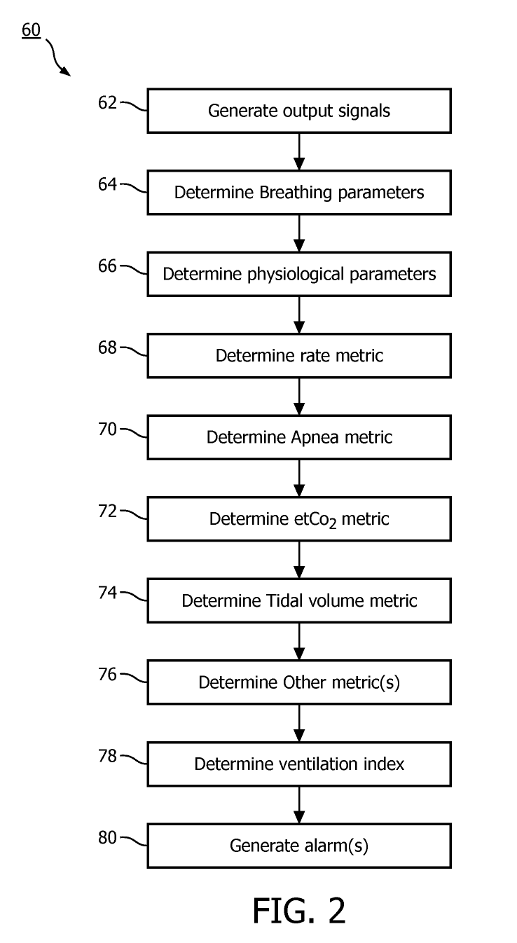

FIG. 2 illustrates a method 60 of monitoring respiration of a subject. The operations of method 60 presented below are intended to be illustrative. In some embodiments, method 60 may be accomplished with one or more additional operations not described, and/or without one or more of the operations discussed. Additionally, the order in which the operations of method 60 are illustrated in FIG. 2 and described below is not intended to be limiting.

In some embodiments, method 60 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 60 in response to instructions stored electronically on an electronic storage medium. The one or more processing devices may include one or more devices configured through hardware, firmware, and/or software to be specifically designed for execution of one or more of the operations of method 60.

At an operation 62, output signals are generated that convey information related to (i) one or more gas parameters of a flow of breathable gas being communicated with the airway of the subject, and/or (ii) one or more physiological parameters of the subject. In some embodiments, operation 62 is performed by one or more sensors the same as or similar to gas parameter sensors 20 and/or physiological sensors 22 (shown in FIG. 1 and described herein).

At an operation 64, breathing parameters of the subject are determined based on the output signals. The breathing parameters include a first parameter related to a rate of respiration of the subject (e.g., breath time and/or respiratory rate), a second parameter related to end tidal carbon dioxide of the subject, a third parameter related to tidal volume of the subject, and/or other parameters. In some embodiments, operation 64 is performed by a breathing parameter module the same as or similar to breathing parameter module 36 (shown in FIG. 1 and described herein). In some embodiments, operation 64 includes determining gas parameters of the flow of breathable gas based on the output signals, and then determining the breathing parameters based on the determined gas parameters.

At an operation 66, one or more physiological parameters are determined based on the output signals. The one or more physiological parameters may not be determined from the output signals conveying information related to the flow of breathable gas. In some embodiments, operation 66 is performed by a physiological parameter module the same as or similar to physiological parameter module 38 (shown in FIG. 1 and described herein).

At an operation 68, a rate metric is determined that indicates deviation from a previous normal respiratory rate by the subject. The rate metric is determined based on a comparison of the first parameter for a first set of breaths by the subject with the first parameter for a first subset of one or more breaths. The first subset of one or more breaths (e.g., a single breath) are also in the first set of breaths. This comparison may include comparing the first parameter for the first subset of one or more breaths with the average and/or standard deviation of the first parameter for the first set of breaths. In some embodiments, operation 68 is performed by a respiratory rate monitor module the same as or similar to respiratory rate monitor module 40 (shown in FIG. 1 and described herein).

At an operation 70, an apnea metric is determined based on the generated output signals and/or the determined parameters. The apnea metric represents whether the subject is currently experiencing and apnea. Responsive to the subject experiencing an apnea, the apnea metric may indicate a severity and/or duration of the apnea. In some embodiments, operation 70 is performed by a apnea monitor module the same as or similar to apnea monitor module 42 (shown in FIG. 1 and described herein).

At an operation 72, an end tidal carbon dioxide metric is determined The end tidal carbon dioxide metric indicates deviations of end tidal carbon dioxide during respiration by the subject from levels that are normal or typical for the subject. The end tidal carbon dioxide metric is determined based on a comparison of the second parameter for a second set of breaths by the subject with the second parameter for a second subset of one or more breaths. The second subset of one or more breaths (e.g., a single breath) are also in the second set of breaths. The second set of breaths may be the same as or different from the first set of breaths. The second subset of one or more breaths may be the same as or different from first subset of one or more breaths. This comparison may include comparing the second parameter for the second subset of one or more breaths with the average and/or standard deviation of the second parameter for the second set of breaths. In some embodiments, operation 72 is performed by an end tidal carbon dioxide monitor module the same as or similar to end tidal carbon dioxide monitor module 44 (shown in FIG. 1 and described herein).

At an operation 74, a tidal volume metric is determined. The tidal volume metric indicates deviations of the subject's tidal volume from levels that are typical or normal for the subject. The tidal volume metric is determined based on a comparison of the third parameter for a third set of breaths by the subject with the third parameter for a third subset of one or more breaths. The third subset of one or more breaths (e.g., a single breath) are also in the third set of breaths. The third set of breaths may be the same as or different from the first and/or second set(s) of breaths. The third subset of one or more breaths may be the same as or different from the first and/or second subset(s) of one or more breaths. This comparison may include comparing the third parameter for the third subset of one or more breaths with the average and/or standard deviation of the third parameter for the third set of breaths. In some embodiments, operation 74 is performed by a tidal volume monitor module the same as or similar to tidal volume monitor module 46 (shown in FIG. 1 and described herein).

At an operation 76, one or more other metrics are determined. The one or more other metrics are determined based on the output signals generated and/or the parameters determined at one or more of operations 62, 64, and/or 66. In some embodiments, operation 76 is performed by one or more other monitor modules 48 the same as or similar to other monitor modules 48 (shown in FIG. 1 and described herein).

At an operation 78, a ventilation index is determined. The ventilation index represents the respiratory stability and/or effectiveness of the subject. The ventilation index is determined based on one or more of the rate metric, the apnea metric, the end tidal carbon dioxide metric, the tidal volume metric, and/or other metrics or parameters. In some embodiments, operation 78 is performed by a ventilation index module the same as or similar to ventilation index module 50 (shown in FIG. 1 and described herein).

At an operation 80, one or more alarms are generated based on the ventilation index. The one or more alarms may include an alarm that is a warning, an alarm that provides insight with respect to a condition of the subject, an alarm that provides insight with respect to one or more actions to be taken, and/or other alarms. The one or more alarms may include an alarm generated based on a comparison of the ventilation index with a threshold. In some embodiments, operation 80 is performed by an alarm module the same as or similar to alarm module 52 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system configured to monitor respiration of a subject, the system comprising:
one or more gas parameter sensors configured to generate output signals conveying information related to one or more gas parameters in a respiratory circuit, wherein the respiratory circuit comprises a subject interface appliance configured to communicate with the airway of a subject; and
a processor configured to execute computer program modules, the computer program modules comprising:
a breathing parameter module configured to determine breathing parameters of the respiration of the subject based on the output signals, the breathing parameters comprising (i) a first parameter related to breath length, and (ii) a second parameter related to end tidal carbon dioxide;
a respiratory rate monitor module configured to determine, in an ongoing manner, a rate metric based on a comparison of the first parameter for a first set of breaths by the subject with the first parameter for a first subset of one or more breaths, wherein the one or more breaths in the first subset of one or more breaths are also in the first set of breaths by the subject;
an apnea monitor module configured to determine, in an ongoing manner based on the output signals, an apnea metric that represents whether the subject is currently experiencing an apnea, and, responsive to the subject currently experiencing an apnea, a severity and/or duration of the apnea;
an end tidal carbon dioxide monitor module configured to determine, in an ongoing manner, an end tidal carbon dioxide metric based on a comparison of the second parameter for a second set of breaths by the subject with the second parameter for a second subset of one or more breaths, wherein the one or more breaths in the second subset of one or more breaths are also in the second set of breaths by the subject; and
a ventilation index module configured to determine, in an ongoing manner, a ventilation index for the subject based on the rate metric, the apnea metric, and the end tidal carbon dioxide metric, such that the ventilation index at a given time represents respiratory stability and/or effectiveness for the subject at the given time.

2. The system of claim 1, wherein the respiratory rate module and the end tidal carbon dioxide monitor module are configured such that the first set of breaths is the same as the second set of breaths.

3. The system of claim 2, wherein the respiratory rate module and the end tidal carbon dioxide monitor module are configured such that the first subset of one or more breaths is the same as the second set of one or more breaths.

4. The system of claim 1, wherein the computer program modules further comprise an alarm module configured to generate, based on the ventilation index, alarms that indicate instability in the respiration of the subject.

5. The system of claim 4, wherein the alarm module is configured to compare the ventilation index with a score threshold, and to generate the alarms based on this comparison.

6. A method of monitoring respiration of a subject, the method comprising:
receiving output signals conveying information related to one or more gas parameters in a respiratory circuit, wherein the respiratory circuit comprises a non-invasive subject interface appliance configured to non-invasively communicate with the airway of a subject; and
determining breathing parameters of the respiration of the subject based on the output signals, the breathing parameters comprising (i) a first parameter related to breath length, and (ii) a second parameter related to end tidal carbon dioxide;
determining, in an ongoing manner, a rate metric based on a comparison of the first parameter for a first set of breaths by the subject with the first parameter for a first subset of one or more breaths, wherein the one or more breaths in the first subset of one or more breaths are also in the first set of breaths by the subject;
determining, in an ongoing manner based on the output signals, an apnea metric that represents whether the subject is currently experiencing an apnea, and, responsive to the subject currently experiencing an apnea, a severity and/or duration of the apnea;
determining, in an ongoing manner, an end tidal carbon dioxide metric based on a comparison of the second parameter for a second set of breaths by the subject with the second parameter for a second subset of one or more breaths, wherein the one or more breaths in the second subset of one or more breaths are also in the second set of breaths by the subject; and
determining, in an ongoing manner, a ventilation index for the subject based on the rate metric, the apnea metric, and the end tidal carbon dioxide metric, such that the ventilation index at a given time represents respiratory stability and/or effectiveness for the subject at the given time.

7. The method of claim 6, wherein the first set of breaths is the same as the second set of breaths.

8. The method of claim 7, wherein the first subset of one or more breaths is the same as the second set of one or more breaths.

9. The method of claim 6, further comprising generating, based on the ventilation index, alarms that indicate instability in the respiration of the subject.

10. The method of claim 9, further comprising comparing the ventilation index with a score threshold, and wherein the generating the alarms is based on this comparison.

11. A system configured to monitor respiration of a subject, the method comprising:
means for receiving output signals conveying information related to one or more gas parameters in a respiratory circuit, wherein the respiratory circuit comprises a subject interface appliance configured to communicate with the airway of a subject; and
means for determining breathing parameters of the respiration of the subject based on the output signals, the breathing parameters comprising (i) a first parameter related to breath length, and (ii) a second parameter related to end tidal carbon dioxide;
means for determining, in an ongoing manner, a rate metric based on a comparison of the first parameter for a first set of breaths by the subject with the first parameter for a first subset of one or more breaths, wherein the one or more breaths in the first subset of one or more breaths are also in the first set of breaths by the subject;
means for determining, in an ongoing manner based on the output signals, an apnea metric that represents whether the subject is currently experiencing an apnea, and, responsive to the subject currently experiencing an apnea, a severity and/or duration of the apnea;
means for determining, in an ongoing manner, an end tidal carbon dioxide metric based on a comparison of the second parameter for a second set of breaths by the subject with the second parameter for a second subset of one or more breaths, wherein the one or more breaths in the second subset of one or more breaths are also in the second set of breaths by the subject; and
means for determining, in an ongoing manner, a ventilation index for the subject based on the rate metric, the apnea metric, and the end tidal carbon dioxide metric, such that the ventilation index at a given time represents respiratory stability and/or effectiveness for the subject at the given time.

12. The system of claim 11, wherein the first set of breaths is the same as the second set of breaths.

13. The system of claim 12, wherein the first subset of one or more breaths is the same as the second set of one or more breaths.

14. The system of claim 11, further comprising means for generating, based on the ventilation index, alarms that indicate instability in the respiration of the subject.

15. The system of claim 14, further comprising means for comparing the ventilation index with a score threshold, and wherein the means for generating is configured to generate the alarms is based on this comparison.

16. The method of claim 1, wherein the end tidal carbon dioxide metric is determined based on a moving window within which an average and a standard deviation of end tidal carbon dioxide are determined for the second subset of breaths taken within the moving window of time.

17. The system of claim 11, wherein the end tidal carbon dioxide metric is determined based on a moving window within which an average and a standard deviation of end tidal carbon dioxide are determined for the second subset of breaths taken within the moving window of time.

18. The method of claim 1, wherein the end tidal carbon dioxide metric indicates a deviation of the end tidal carbon dioxide from a typical end tidal carbon dioxide volume of the subject.

19. The system of claim 1, wherein the end tidal carbon dioxide metric indicates a deviation of the end tidal carbon dioxide from a typical end tidal carbon dioxide volume of the subject.
